(19) United States
(12) Patent Application Publication
(10) Pub. No.: US 2025/0135113 A1
(43) Pub. Date: May 1, 2025

(54) FLOW MEASUREMENT DEVICES FOR CANCER THERAPY SYSTEMS

(71) Applicant: Boston Scientific Medical Device Limited, Galway (IE)

(72) Inventors: Nachiket Gole, Pune (IN); Nitesh Ghananil Baviskar, Kalyan West (IN); Mihir Sukhatme, Mumbai (IN); Scott McGhee, Ottawa (CA)

(21) Appl. No.: 18/930,622

(22) Filed: Oct. 29, 2024

Related U.S. Application Data
(60) Provisional application No. 63/546,359, filed on Oct. 30, 2023.

(51) Int. Cl. A61M 5/168; A61M 5/178; A61M 39/24

(52) U.S. Cl. CPC A61M 5/16886; A61M 5/178; A61M 39/24; A61M 2205/583

(57) ABSTRACT

Embodiments herein relate to flow measurement devices used with cancer therapy systems and related methods. In an embodiment, a flow measurement device for cancer therapy is included having a housing that defines an internal volume along with an ingress port and an egress port. The flow measurement device can further include a sliding marker, wherein the sliding marker is configured to slide within the internal volume of the housing. The sliding marker can be mechanically biased in a first direction in the absence of fluid flow through the internal volume of the housing then slide in a second direction opposite the first direction as a result of fluid flow through the internal volume of the housing. The position of the sliding marker can be indicative of the fluid flow rate through the flow measurement device. Other embodiments are also included herein.

This application claims the benefit of U.S. Provisional Application No. 63/546,359, filed Oct. 30, 2023, the content of which is herein incorporated by reference in its entirety.

### FIELD

Embodiments herein relate to flow measurement devices used with cancer therapy systems and related methods.

### BACKGROUND

According to the American Cancer Society, cancer accounts for nearly 25% of the deaths that occur in the United States each year. Cancerous tumors can form if one normal cell in any part of the body mutates and then begins to grow and multiply too rapidly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division. Surgery is a common first-line therapy for many cancerous tumors. However, not every tumor can be surgically removed.

### SUMMARY

Embodiments herein relate to flow measurement devices used with cancer therapy systems and related methods. In a first aspect, a flow measurement device for cancer therapy is included having a housing that defines an internal volume along with an ingress port and an egress port. The flow measurement device can further include a sliding marker, wherein the sliding marker is configured to slide within the internal volume of the housing. The sliding marker can be mechanically biased in a first direction in the absence of fluid flow through the internal volume of the housing then slide in a second direction opposite the first direction as a result of fluid flow through the internal volume of the housing. The position of the sliding marker can be indicative of the fluid flow rate through the flow measurement device.

In a second aspect, in addition to one or more of the preceding or following aspects, or in the alternative to some aspects, the ingress port is configured to be in fluid communication with a fluid delivery device.

In a third aspect, in addition to one or more of the preceding or following aspects, or in the alternative to some aspects, the fluid delivery device can include a syringe.

In a fourth aspect, in addition to one or more of the preceding or following aspects, or in the alternative to some aspects, a cancer therapy system can further include a valve, wherein the valve can be disposed between the housing and the fluid delivery device.

In a fifth aspect, in addition to one or more of the preceding or following aspects, or in the alternative to some aspects, the internal volume of the flow measurement device can be substantially cylindrical in cross-section.

In a sixth aspect, in addition to one or more of the preceding or following aspects, or in the alternative to some aspects, the internal volume of the flow measurement device can be tapered such that a cross-sectional area of the internal volume changes along a length of the housing.

In a seventh aspect, in addition to one or more of the preceding or following aspects, or in the alternative to some aspects, the sliding marker can include an aperture and the flow measurement device can further include a guide rod, wherein the guide rod can be disposed within the internal volume, and wherein the guide rod passes through the aperture of the sliding marker.

In an eighth aspect, in addition to one or more of the preceding or following aspects, or in the alternative to some aspects, the flow measurement device can further include a spring, wherein the spring can be used to mechanically bias the sliding marker in the first direction.

In a ninth aspect, in addition to one or more of the preceding or following aspects, or in the alternative to some aspects, indicia (such as markings) can be disposed on a surface of the housing indicating flow rates and a current flow rate can be determined based on identifying where the sliding marker lines up with the indicia.

In a tenth aspect, in addition to one or more of the preceding or following aspects, or in the alternative to some aspects, the housing of the flow measurement device can be transparent.

In an eleventh aspect, in addition to one or more of the preceding or following aspects, or in the alternative to some aspects, the flow measurement device can further include one or more projections, wherein the one or more projections can be arranged within the internal volume to limit travel of the sliding marker.

In a twelfth aspect, a flow measurement device for cancer therapy can be included having a housing that defines an internal volume along with an ingress port, a side egress port, and a sliding piston. The sliding piston can be configured to slide within the internal volume of the housing. The sliding piston can be mechanically biased in a first direction then slide in a second direction opposite the first as a result of fluid flow through the internal volume of the housing. The sliding piston occludes the side egress port when slid in the first direction then opens the side egress port when slid in the second direction functioning as a valve. Also, the position of the sliding piston can be indicative of fluid flow rate through the flow measurement device for cancer therapy.

In a thirteenth aspect, in addition to one or more of the preceding or following aspects, or in the alternative to some aspects, the ingress port is configured to be in fluid communication with a fluid delivery device.

In a fourteenth aspect, in addition to one or more of the preceding or following aspects, or in the alternative to some aspects, the fluid delivery device can include a syringe.

In a fifteenth aspect, in addition to one or more of the preceding or following aspects, or in the alternative to some aspects, the housing can be substantially cylindrical in cross-section.

In a sixteenth aspect, in addition to one or more of the preceding or following aspects, or in the alternative to some aspects, the flow measurement device can further include a spring, wherein the spring can be used to mechanically bias the sliding piston in the first direction.

In a seventeenth aspect, in addition to one or more of the preceding or following aspects, or in the alternative to some aspects, indicia (such as markings) can be disposed on a surface of the housing indicating flow rates and a current flow rate can be determined based on identifying where the sliding piston lines up with the indicia.

In an eighteenth aspect, in addition to one or more of the preceding or following aspects, or in the alternative to some aspects, the housing can be transparent.

In a nineteenth aspect, in addition to one or more of the preceding or following aspects, or in the alternative to some aspects, the housing can define a side port, wherein the sliding piston occludes fluid communication with the side port when fully biased in the first direction and opens fluid communication with the side port when slid in the second direction.

In a twentieth aspect, in addition to one or more of the preceding or following aspects, or in the alternative to some aspects, further can include one or more projections, wherein the one or more projections can be arranged within the internal volume to limit travel of the sliding piston.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a diagram of a cancer treatment system in accordance with various embodiments herein.

FIG. 2 is a schematic view of a flow measurement device in accordance with various embodiments herein.

FIG. 3 is a schematic view of a flow measurement device in accordance with various embodiments herein.

FIG. 4 is a schematic view of a flow measurement device in accordance with various embodiments herein.

FIG. 5 is a schematic view of a flow measurement device in accordance with various embodiments herein.

FIG. 6 is a schematic view of a flow measurement device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, surgery is a common first-line therapy for many cancerous tumors. However, not every tumor can be surgically removed. Another approach to treating some types of cancer can include delivering radiation to cancerous tissue to destroy cancerous cells therein. In one approach, microspheres such as small glass microspheres that have Y-90 mixed into the glass can be deposited in or near a cancerous tumor and beta radiation emitting therefrom can damage DNA of the cancerous cells inducing apoptosis and cell cycle arrest.

Delivery of microspheres can be performed using a catheter, such as a microcatheter placed in vasculature leading to a tumor, such as an artery supplying blood to the tumor. The microspheres can be combined with a fluid, such as saline, to form a suspension that is then directed through the catheter to the site of deposition.

In some approaches, a clinician must utilize a syringe or similar device to deliver the fluid to be mixed with the microspheres. The clinician advances the plunger of the syringe at a desired pressure/speed resulting in a particular flow rate of fluid through the system for a particular amount of time to a achieve a target volume of suspension delivered out of the catheter and into the patient. However, it is tedious for system users to manage aspects of this process. For example, it is challenging to accurately determine the actual flow rate achieved relative to targeted bounds (such as from 5 milliliters (ml)/min to 20 ml/min, or within other bounds).

Systems and methods herein can be used to accurately measure flow rates of fluid from fluid delivery devices. Advantageously, flow rate can be measured using purely mechanical means which prevents the need for more complicated systems requiring a power source. In an embodiment, a flow measurement device, such as a flow measurement device for cancer therapy is included having a housing that defines an internal volume along with an ingress port and an egress port. A sliding marker element can be configured to slide within the internal volume of the housing. The sliding marker can be mechanically biased in a first direction (such as with a spring) in the absence of fluid flow through the internal volume of the housing. Then the sliding marker can slide in a second direction (opposite the first direction) as a result of fluid flow through the internal volume of the housing. The greater the flow rate, the farther the sliding marker moves in the second direction. Markings or other indicia can be disposed on the housing, at least a portion of which can be transparent. The markings or other indicia can correspond to different flow rates. Thus, a user can see the position of the sliding marker element within the housing and based on the markings or indicia can determine the flow rate based on the position of the sliding marker element. The sliding marker can have various shapes. In some embodiments, the sliding marker can be cylindrical, conical, cuboidal, irregular, or spherical, amongst others. In some embodiments, the sliding marker can be hollow. In some embodiments, the sliding marker can be solid. Various materials can be used to form the sliding marker including, but not limited to, polymers, composites, glasses, metals, natural materials, and the like.

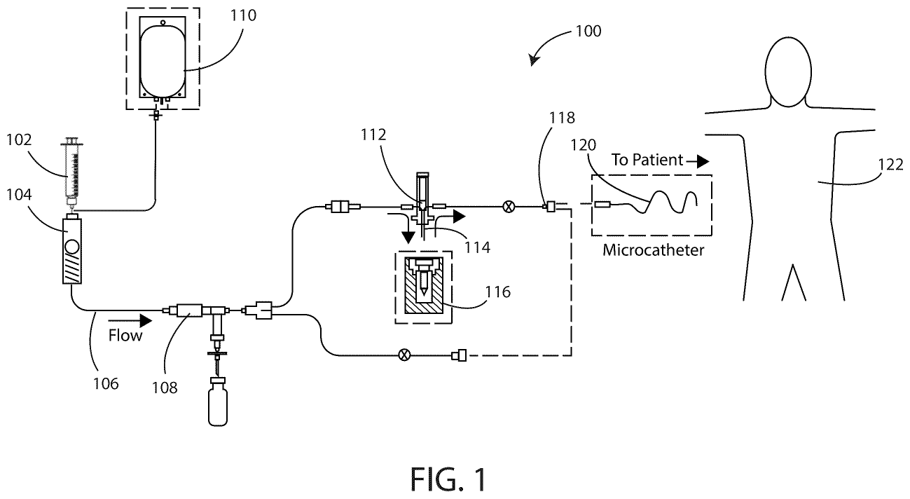

Referring now to FIG. 1, a diagram of a cancer treatment system 100 is shown in accordance with various embodiments herein. Major parts of the cancer treatment system 100 include a therapeutic fluid delivery device 102, a flow measurement device 104, a fluid supply tube 106, and a flow control valve 108. The cancer treatment system 100 also includes a saline supply reservoir 110. The cancer treatment system 100 also includes a fluid injection and suspension withdrawal device 112. The cancer treatment system 100 also includes a radioactive microsphere supply reservoir 116. The cancer treatment system 100 also includes an outflow port 118. The cancer treatment system 100 also includes a microcatheter 120. FIG. 1 shows a patient 122 in which the microcatheter 120 can be inserted to deliver the therapeutic suspension of microspheres.

In use, (omitting some possible operations as well as some components for ease of explanation) the clinician or other system user can pull back on a plunger or similar mechanism of therapeutic fluid delivery device 102 causing fluid (such as saline) to be withdrawn from the saline supply reservoir 110 and into the fluid delivery device 102. Then the clinician or other system user can depress the plunger causing fluid to flow from the therapeutic fluid delivery device 102, through the flow measurement device 104, through the fluid supply tube 106, through the flow control valve 108, and into the fluid injection and suspension withdrawal device 112. The fluid injection and suspension withdrawal device 112 can be in fluid communication with the radioactive microsphere supply reservoir 116 and can direct a flow of fluid into the radioactive microsphere supply reservoir 116 coming from the therapeutic fluid delivery device 102 such as through one of a pair of needles, cannulas, or tubes 114. The fluid can become mixed with microspheres in the radioactive microsphere supply reservoir 116 forming a suspension which can then exit via the fluid injection and suspension withdrawal device 112 via another needle, cannula, or tube 114 and through tubing and out of the outflow port 118 and into the microcatheter 120 before passing to a desired site of the patient 122. It will be appreciated that various other operations can also be performed including, but not limited to, system priming, bubble removal, one or more flushing operations, and the like.

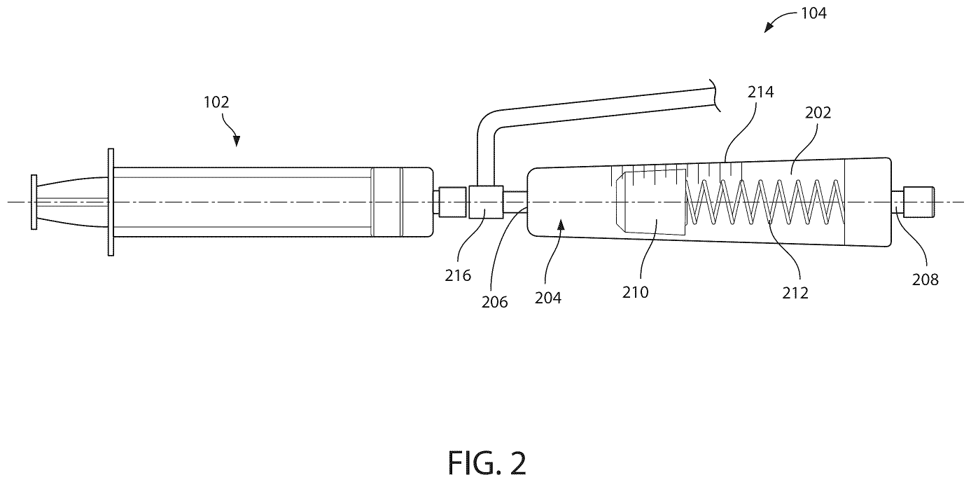

Referring now to FIG. 2, a schematic view of a flow measurement device 104 is shown in accordance with various embodiments herein. FIG. 2 shows the flow measurement device 104 in fluid communication with fluid delivery device 102. The flow measurement device 104 includes a housing 202 defining an internal volume 204 and a sliding marker 210 disposed therein. The flow measurement device 104 also includes an ingress port 206 and an egress port 208. In some embodiments, the housing 202 can be formed of a transparent material (such as a polymer or a glass) to allow the position of the sliding marker 210 to be visible from outside of the housing 202. The flow measurement device 104 also includes a spring 212 (or similar mechanical element) which can be formed of a metal or another material suitable for generating a spring force such as some types of polymers or composites. The spring 212 can be used to provide a biasing force on the sliding marker 210. For example, the spring 212 can cause the sliding marker 210 to be biased in a first direction (an upstream direction). When fully biased in the first direction (such as when there is no flow through the flow measurement device 104), the end of the sliding marker 210 can be lined up with indicia 214 that corresponds to a flow rate of 0 ml/min (or no flow).

In this embodiment, a valve 216 is also included and disposed between the fluid delivery device 102 and the flow measurement device 104. The valve 216 can provide selective fluid communication with the saline (or other fluid) supply reservoir 110.

In operation, a clinician or device operator can pull the plunger back on the fluid delivery device 102 which can cause a fluid to flow from the supply reservoir 110, through the valve 216, and into the barrel of the fluid delivery device 102 while bypassing the flow measurement device 104. However, in some embodiments, the valve 216 can be arranged downstream from the flow measurement device 104 such that fluid passes through flow measurement device 104 even when fluid is being drawn into the fluid delivery device 102 from the supply reservoir 110.

After the fluid delivery device 102 is filled with fluid, then the clinician or device operator can begin to depress the plunger of the fluid delivery device 102 causing fluid within the fluid delivery device 102 to be expelled and passing through the valve 216 and into the flow measurement device 104. The faster the flow rate, the more force is applied by the fluid to the sliding marker 210. This force from fluid flow can overcome the biasing force of the spring 212, causing the sliding marker 210 to move in the direction of the fluid flow. Thus, there is a relationship between the flow rate and the position of the sliding marker 210. The position of the sliding marker 210 can then be used to determine flow rate by referencing the position of the sliding marker 210 with respect to indicia 214. For example, the system can be configured so that the force generated by certain flow rates causes the sliding marker 210 to line up with the indicia 214 that accurately describe the current flow rate for the clinician or device operator. The indicia 214 (or markings) are typically disposed on or adjacent to a surface of the housing 202 indicating flow rates and a current flow rate can be determined based on identifying where the sliding marker 210 lines up with the indicia 214.

In various embodiments, the internal volume 204 of the housing 202 is substantially cylindrical in cross-section. However, the internal volume 204 could also have other shapes in cross-section. In various embodiments, the internal volume 204 of the housing 202 can be tapered such that a cross-sectional area of the internal volume 204 changes along a length of a housing 202. For example, the taper can be such that the cross-sectional area of the internal volume 204 is larger at a downstream position versus an upstream position. In such a case, the frictional force of fluid passing by the sliding marker 210 can change based on the position of the sliding marker 210 within the flow measurement device 104 as a perimeter gap between the sliding marker 210 and an inner surface of the internal volume 204 will change depending on the current position of the sliding marker 210 along the lengthwise axis of the internal volume 204.

In various embodiments, the fluid delivery device 102 can include a syringe. However, the flow measurement device 104 herein can work with other types of fluid delivery devices including both manually actuated and powered fluid delivery devices.

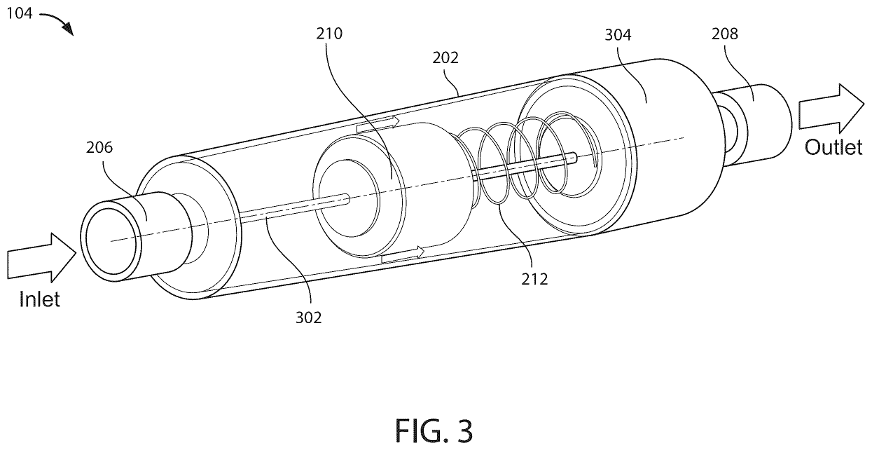

Referring now to FIG. 3, a schematic view of a flow measurement device is shown in accordance with various embodiments herein. FIG. 3 shows a flow measurement device 104, as before, including a housing 202, a sliding marker 210 therein, and a spring 212 to provide a biasing force on the sliding marker 210. FIG. 3 also shows an ingress port 206 and an egress port 208. In the embodiment of FIG. 3, the flow measurement device 104 also includes a guide rod 302. The flow measurement device 104 also includes a base structure 304.

The guide rod 302 can be disposed within the internal volume 204 of the flow measurement device 104 and can service to guide the sliding marker 210 as it slides within the internal volume 204. This can prevent it from turning within the internal volume 204 far enough to interface with the inner surface of the internal volume 204 in a manner that would cause the force required to move the sliding marker 210 to vary enough to make the flow measurements inaccurate. The guide rod 302 can be secured to the housing 202 at points upstream and downstream of where the sliding marker 210 must slide within the internal volume 204.

In various embodiments, the sliding marker 210 can include an aperture to allow passage of the guide rod 302 therethrough. In various embodiments, the aperture to receive the guide rod 302 is substantially circular, however it could also take on various other shapes while still be sufficient to allow passage of the guide rod 302 therethrough.

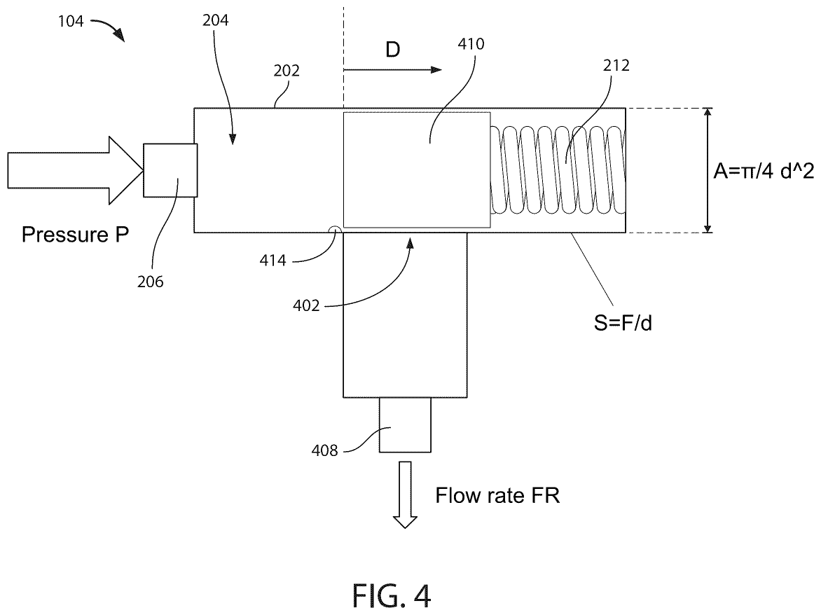

In some embodiments, movement of an element within the internal volume 204 of the flow measurement device 104 can serve as both a measurement device as well as a valve (such as a one-way valve). Referring now to FIG. 4, a schematic view of a flow measurement device 104 is shown in accordance with various embodiments herein. As before, the flow measurement device 104 includes a housing 202, an internal volume 204, an ingress port 206, and a spring 212. However, in this embodiment, the flow measurement device 104 also includes a side egress port 408 and a sliding piston 410. The side egress port 408 can provide a fluid flow path onto other downstream components of the cancer treatment system 100 such as the fluid injection and suspension withdrawal device 112, radioactive microsphere supply reservoir 116, outflow port 118, and microcatheter 120.

In various embodiments, the sliding piston 410 can be configured to slide within an internal volume 204 of a housing 202. The sliding piston 410 can be mechanically biased in a first direction and slide in a second direction opposite the first as a result of fluid flow through an internal volume 204 of a housing 202. In some embodiments, a structural feature can be included to allow fluid in the area of the spring 212 to be displaced as the sliding piston 410 slides in the second direction (compressing the spring 212). For example, the area of the chamber in the area of the spring 212 can be vented to a fillable component such as an expandable or vented reservoir, or vented to the atmosphere. In various embodiments, the sliding piston 410 occludes a side egress port 408 when slid sufficiently far in a first direction (a closed position for the sliding piston 410). Then when fluid enters the internal volume 204 under pressure as driven by a fluid delivery device, such force can overcome the biasing force of the spring 212 causing the sliding piston 410 to move in a second direction opposite the first direction and allowing fluid to flow to the side 402 and out the side egress port 408. Further, the degree of movement of the sliding piston 410 is dependent on pressure driving a particular flow rate and therefore the position of the sliding piston 410 is indicative of fluid flow rate through the flow measurement device for cancer therapy 104. Thus, a current flow rate can be determined based on identifying where the sliding piston 410 lines up with the indicia 214. As such, the sliding piston 410 can serve dual functions as a valve mechanism and also an indicator of fluid flow rate for a given pressure difference between upstream and downstream across the device.

In various embodiments, the housing 202 include one or more projections 414 or similar elements to limit the travel of the sliding piston 410 within the internal volume 204.

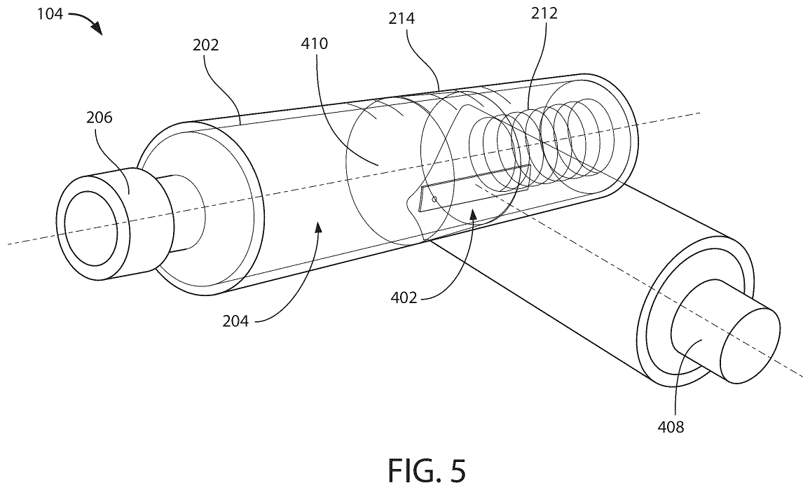

Referring now to FIG. 5, another schematic view of a flow measurement device 104 is shown in accordance with various embodiments herein. As with the embodiment shown in FIG. 4, the flow measurement device 104 includes a housing 202, an internal volume 204, an ingress port 206, a spring 212, a side 402, a side egress port 408 and a sliding piston 410. FIG. 5 also shows indicia 214 disposed on or in a surface of the housing 202.

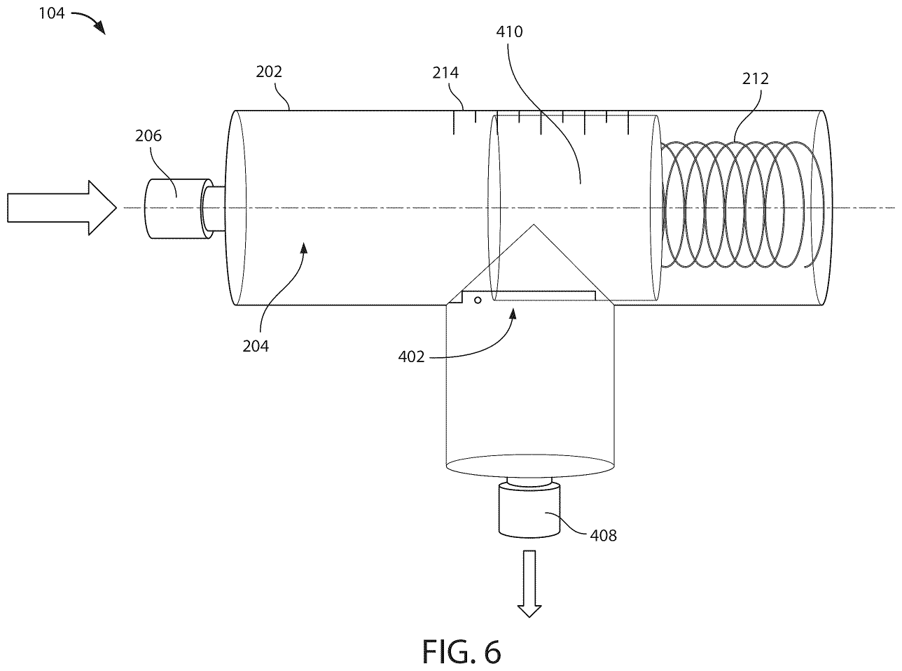

Referring now to FIG. 6, another schematic view of a flow measurement device is shown in accordance with various embodiments herein. As with the embodiment shown in FIG. 5, the flow measurement device 104 includes a housing 202, an internal volume 204, an ingress port 206, a spring 212, a side 402, and a side egress port 408, sliding piston 410, and indicia 214 disposed on or in a surface of the housing 202.

### Methods

Many different methods are contemplated herein, including, but not limited to, methods of making, methods of using, and the like. Aspects of system/device operation described elsewhere herein can be performed as operations of one or more methods in accordance with various embodiments herein.

In some embodiments, a method of measuring a flow rate of a cancer treatment system 100 using only mechanical components is included. The method can include connecting a flow measurement device as described herein to a fluid delivery device such as a syringe or the like. The method can include drawing fluid into the fluid delivery device from a supply reservoir, in some cases bypassing the flow measurement device. The method can further include actuating the fluid delivery device (such as depressing a plunger of the fluid delivery device) causing fluid to flow from the fluid delivery device to the flow measurement device. The method can further include monitoring a position of a marker element and/or a sliding piston to monitor the flow rate of the fluid and modulating a flow rate produced by the fluid delivery device to maintain it within target bounds.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A flow measurement device for cancer therapy comprising:
a housing, wherein the housing defines an internal volume along with an ingress port and an egress port; and
a sliding marker;
wherein the sliding marker is configured to slide within the internal volume of the housing; and
wherein the sliding marker is mechanically biased in a first direction in the absence of fluid flow through the internal volume of the housing then slides in a second direction opposite the first as a result of fluid flow through the internal volume of the housing.

2. The flow measurement device for cancer therapy of claim 1, wherein the ingress port is configured to be in fluid communication with a fluid delivery device.

3. The flow measurement device for cancer therapy of claim 2, the fluid delivery device comprising a syringe.

4. The flow measurement device for cancer therapy of claim 2, further comprising a non-return valve, wherein the non-return valve is disposed between the housing and the fluid delivery device.

5. The flow measurement device for cancer therapy of claim 1, wherein the internal volume is substantially cylindrical in cross-section.

6. The flow measurement device for cancer therapy of claim 1, wherein the internal volume is tapered such that a cross-sectional area of the internal volume changes along a length of the housing.

7. The flow measurement device for cancer therapy of claim 1, the sliding marker comprising:
an aperture;
the flow measurement device for cancer therapy further comprising a guide rod, wherein the guide rod is disposed within the internal volume; and
wherein the guide rod passes through the aperture.

8. The flow measurement device for cancer therapy of claim 1, further comprising a spring, wherein the spring is used to mechanically bias the sliding marker in the first direction.

9. The flow measurement device for cancer therapy of claim 1, wherein indicia are disposed on a surface of the housing indicating flow rates and a current flow rate can be determined based on identifying where the sliding marker lines up with the indicia.

10. The flow measurement device for cancer therapy of claim 1, wherein the housing is transparent.

11. The flow measurement device for cancer therapy of claim 1, further comprising one or more projections, wherein the one or more projections are arranged within the internal volume to limit travel of the sliding marker.

12. A flow measurement device for cancer therapy comprising:
a housing, wherein the housing defines an internal volume along with an ingress port and a side egress port; and
a sliding piston;
wherein the sliding piston is configured to slide within the internal volume of the housing;
wherein the sliding piston is mechanically biased in a first direction then slides in a second direction opposite the first as a result of fluid flow through the internal volume of the housing;
wherein the sliding piston occludes the side egress port when slid in the first direction then opens the side egress port when slid in the second direction; and
wherein the position of the sliding piston is indicative of fluid flow rate through the flow measurement device for cancer therapy.

13. The flow measurement device for cancer therapy of claim 12, wherein the ingress port is configured to be in fluid communication with a fluid delivery device.

14. The flow measurement device for cancer therapy of claim 13, the fluid delivery device comprising a syringe.

15. The flow measurement device for cancer therapy of claim 12, wherein the housing is substantially cylindrical in cross-section.

16. The flow measurement device for cancer therapy of claim 12, further comprising a spring, wherein the spring is used to mechanically bias the sliding piston in the first direction.

17. The flow measurement device for cancer therapy of claim 12, wherein indicia are disposed on a surface of the housing indicating flow rates and a current flow rate can be determined based on identifying where the sliding piston lines up with the indicia.

18. The flow measurement device for cancer therapy of claim 12, wherein the housing is transparent.

19. The flow measurement device for cancer therapy of claim 12, the housing defining a side port;
wherein the sliding piston occludes the side port when fully biased in the first direction and opens the side port when slid in the second direction.

20. The flow measurement device for cancer therapy of claim 12, further comprising one or more projections, wherein the one or more projections are arranged within the internal volume to limit travel of the sliding piston.
